(19) United States
(12) Patent Application Publication
(10) Pub. No.: US 2003/0012680 A1
(43) Pub. Date: Jan. 16, 2003

(54) Deodorizer/odorizer device

(76) Inventor: Richard Balsys, Malibu, CA (US)

Correspondence Address: KELLY BAUERSFELD LOWRY & KELLEY, LLP, 6320 CANOGA AVENUE, SUITE 1650, WOODLAND HILLS, CA, 91367, US

(21) Appl. No.: 10/197,966

(22) Filed: Jul. 16, 2002

Related U.S. Application Data
(60) Provisional application No. 60/305,613, filed on Jul. 16, 2001.

(51) Int. Cl. A61L 9/00; B32B 5/02

(52) U.S. Cl. 422/5; 422/4; 422/120; 422/123; 422/124

(57) ABSTRACT

An odorizer/deodorizer device includes two shells forming a housing defining an internal cavity. A scented insert is disposed within the housing. The insert may be an absorbent pad containing a liquid-based scent material. An end of the housing is configured such so as to be securely positioned in alignment with a fan outlet of an electrical device. The housing may include a tab having an aperture which receives a hook attachable to the fan or electrical device for positioning the housing relative to the fan. The housing includes vents to allow air to be forced therethrough. The scented insert may also include apertures to facilitate the flow of air therethrough so that as the fan is in operation, the air forced therethrough is odorize or deodorized.

### RELATED APPLICATION

This application claims priority from U.S. Provisional Application No. 60/305,613 filed Jul. 16, 2001.

### BACKGROUND OF THE INVENTION

The present invention relates to room deodorizers, and sometimes referred to as odorizers. More particularly, the present invention relates to room deodorizers that attach to the fan of an electronic device, such as a computer.

Air fresheners or other scented devices are well known to consumers. Traditional air fresheners or deodorizing devices, however, have certain drawbacks. Because air flow through a traditional deodorizing device is passive, the device needs to be placed in open air for appropriate circulation. Often times, however, these deodorizing devices are unsightly and most people would want them concealed. Some deodorizing devices plug into an electrical outlet and use electricity to provide better circulation of the deodorant or air freshener. These devices still do not provide adequate circulation and they further inconvenience the consumer by occupying an electrical outlet.

Many offices, homes, cars and or other rooms have electronic devices such as computers or power amplifiers that require a fan to prevent overheating of the electrical device. Because of the forced air throughput of these fans, the air leaving the fan is circulated throughout the room or surrounding environment. Heretofore, such fans of electronic devices have not been utilized in association with air fresheners or other scented devices. However, associating an air freshener or deodorizer with an electronic device a fan would provide enhanced disbursement of the scent or deodorizer. Also, the deodorizing device would be placed in a convenient and concealed location without the need to occupy an electrical outlet of the room.

Accordingly, there is a need for a deodorizer that can attach to the fan of an electrical device and use the air flow of the fan to freshen or deodorize a room. The present invention fulfills these needs and provides other related advantages.

### SUMMARY OF THE INVENTION

The present invention resides in an odorizer/deodorizer device configured to attach to a fan, such as those found on electronic devices, and allow air to flow therethrough whereby air flowing through the device is scented, refreshed or deodorized.

The device generally comprises a housing defining an internal cavity. Typically, the housing comprises two shells removably attachable to one another. In a particularly preferred embodiment, the two shells are threadably attached to one another.

An end of the housing is configured such so as to be securely positioned in alignment with a fan outlet of an electrical device. The housing has vents to allow air to be forced therethrough. In a particularly preferred embodiment, opposing ends of the housing in alignment with the fan outlet are comprised of a mesh material.

A scented insert is disposed within the housing, such that when air is forced through the housing by the fan the air is odorized or deodorized. The insert may comprise an absorbent pad containing a liquid-based scent material. In order to not impede the flow of air from the electronic device, the insert preferably includes a plurality of apertures to facilitate the flow of air therethrough.

In use, a scented insert is placed into the housing. The housing is then securely positioned in alignment with a fan outlet of an electrical device. Typically, a tab extends from the housing and includes an aperture for connection to the fan or electrical device. A hook may be attachable to the fan or electrical device and insertable through the aperture of the tab for positioning the housing relative to the fan. Thus, when the electrical device is powered, air is forced through the housing and scented insert by the fan causing the air to be odorized or deodorized.

The invention may be offered as a kit including the housing comprised of two shells removably attachable to one another so as to define an internal cavity. An absorbent pad configured to be disposed within the internal cavity of the housing is another component of the kit. A bottle of liquid scent material for selective application to the absorbent pad is also provided. A small hook which is attachable to the fan or electrical device, such as by double-sided tape or adhesive, is also provided to position the housing in alignment with the fan of the electrical device.

Other features and advantages of the present invention will become apparent from the following more detailed description, taken in connection with the accompanying drawings, which illustrate, by way of example, the principles of the invention.

### BRIEF DESCRIPTION OF THE DRAWINGS

The accompanying drawings illustrate the invention. In such drawings:

FIG. 1 is a front perspective view of a odorizer/deodorizer device embodying the present invention;

FIG. 2 is a rear perspective view of the device of FIG. 1;

FIG. 3 is an end view taken generally along line 3-3 of FIG. 1;

FIG. 4 is a cross-sectional view taken generally along line 4-4 of FIG. 1, and illustrating a scented insert within the device;

FIG. 5 is an exploded perspective view of the component parts of the device of the present invention;

FIG. 6 is a perspective view of component parts of a kit of the present invention, illustrating the application of liquid-based scented material to the absorbent pad insert in accordance with the present invention;

FIG. 7 is a perspective view illustrating the attachment of the device of the present invention in alignment with a fan of an electrical device; and

FIG. 8 is a perspective of the device of the present invention attached to the electrical device and illustrating air flowing therethrough.

### DETAILED DESCRIPTION OF THE PREFERRED EMBODIMENTS

The deodorizing device 10 of the present invention provides a simple and convenient method for deodorizing an office or other rooms that have electronic devices such as computers. Computers especially, are becoming commonplace in offices and homes. Furthermore, offices generally do not otherwise have a convenient location for placing a deodorizing device. The deodorizing device 10 allows a person to conveniently use the fan on the back of their desktop computer as a deodorizer or air freshener. The deodorizing device 10 provides superior circulation of deodorant or air freshener without occupying an electrical outlet or being placed in a conspicuous location.

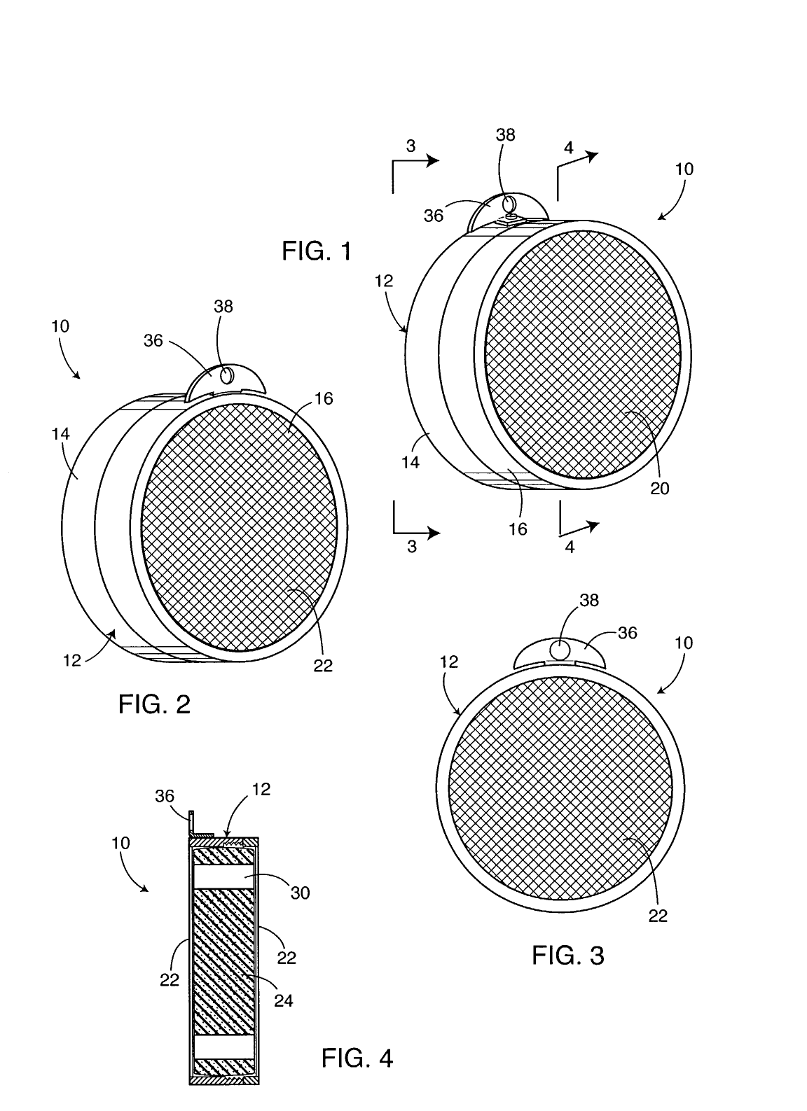

With reference now to FIGS. 1-5, the device 10 of the present invention is comprised of a housing 12 having two halves or shells 14 and 16 removably attachable to one another to form an interior cavity. Although the housing 12 is illustrated as being generally cylindrical, it should be understood that the housing 12 can be of varying sizes and shapes so long as the housing 12 can be positioned in alignment with a fan of an electrical device. Typically, such fans and outlets of electrical devices, such as computers, are generally circular and several inches in diameter. Thus, the preferred cylindrical shape of the housing 12 is illustrated, although the invention is not limited to such.

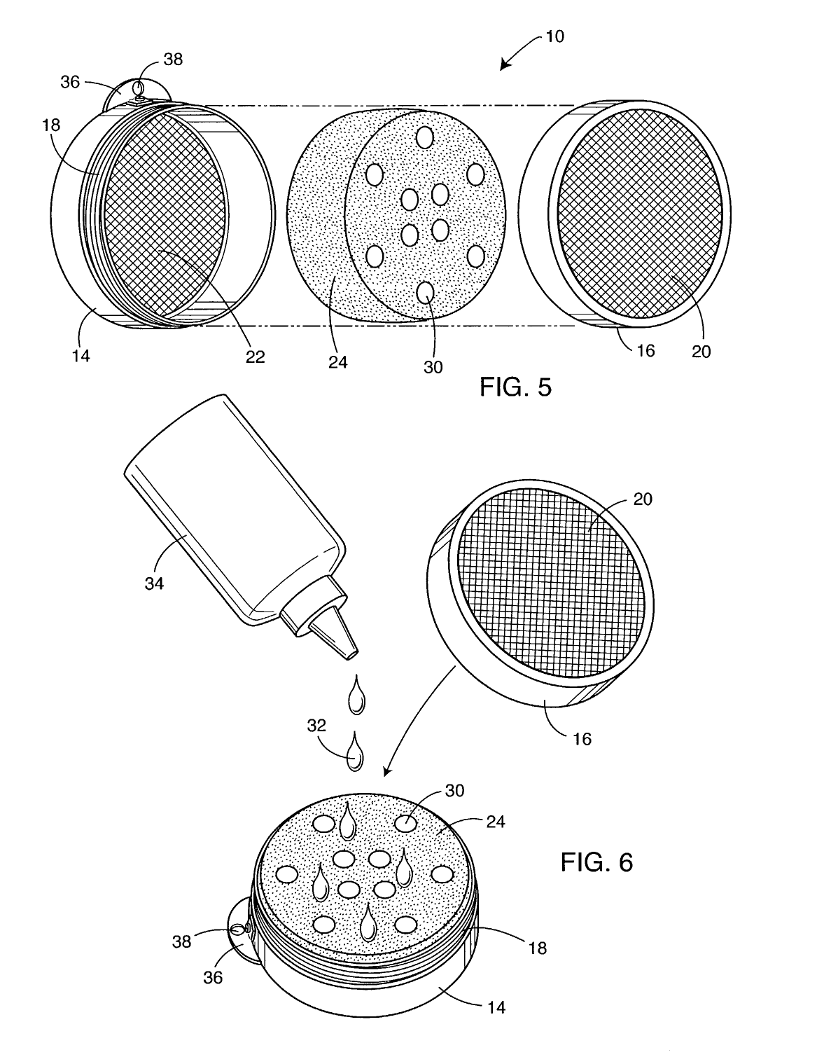

The shells 14 and 16 can be removably attachable to one another by a variety of means. In a particularly preferred embodiment, as illustrated in FIG. 5, one of the shells 14 or 16 includes an area of reduced cross-sectional diameter that includes external threads 18, and the other includes mating threads so that the two shells 14 and 16 can be threadably attached to one another.

The housing 12 includes vents to allow the air from the fan to easily flow therethrough. In a particularly preferred embodiment, as illustrated, ends 20 and 22 of the housing 12 are comprised of a mesh or screen material to facilitate the flow of air through the device 10. It will be appreciated that impeding the flow of air from the electrical device can overheat electrical components and cause damage to the electrical device. Thus, the device 10 of the present invention is intended to freely allow the free flow of air therethrough to the greatest extent possible.

With reference now to FIGS. 4 and 5, a scented insert 24 is disposed within the internal cavity of the housing 12. The insert 24 can be comprised of a variety of materials, such as scented gel material, cotton, fabric, or sponge material. In a particularly preferred embodiment of the present invention, the insert 24 is comprised of an open-cell sponge material which is configured to substantially occupy the internal cavity of the housing 12.

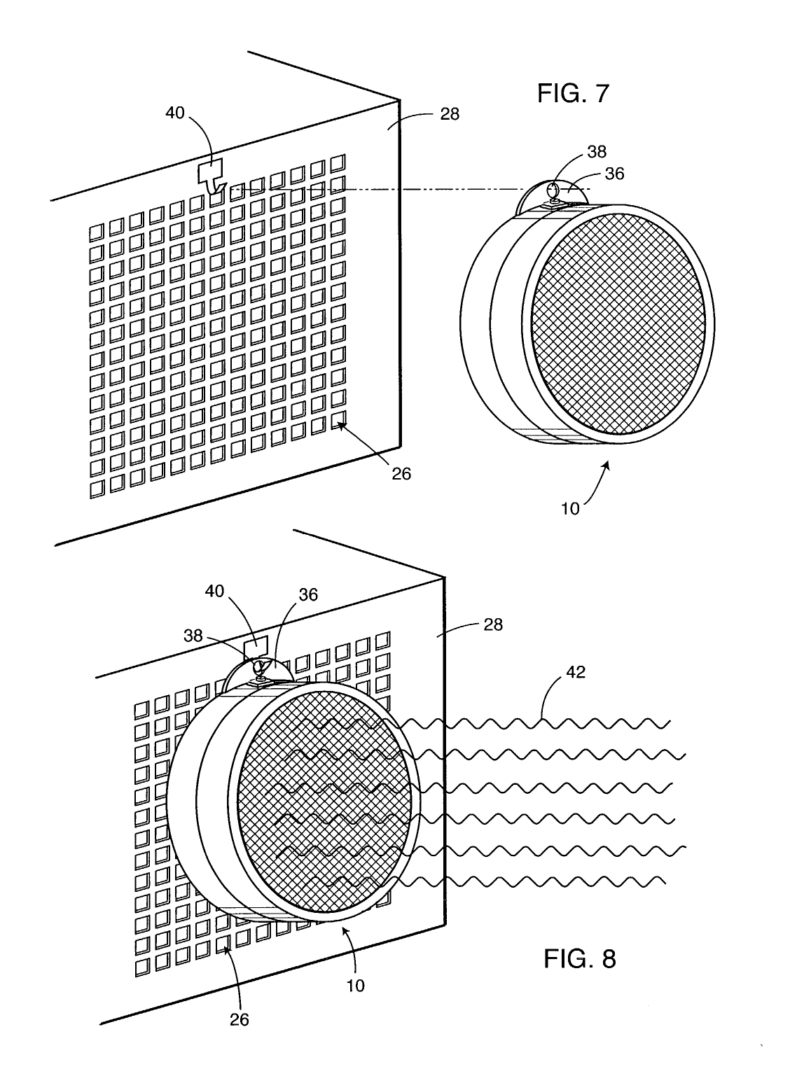

In use, as shown in FIGS. 7 and 8, the insert 24 is disposed within the housing 12 and the device 10 is placed in alignment with an outlet 26 of a fan of an electrical device 28. As the electrical device 28 is powered, the fan forces air through the housing 12 and insert 24 to create a stream of odorized or deodorized air 42.

In order to facilitate the flow of air through the device 10, the insert 24 may include a plurality of apertures 30 therethrough when deemed necessary. Such apertures 30 prevent the impedance of the flow of air through the device 10 and the attending consequences of overheating of the electrical device 28.

With reference now to FIG. 6, the invention may be offered to the consumer in the form of a kit. Such a kit would include the housing 12 comprised of the removably attachable shells 14 and 16. An absorbent pad comprises the insert 24. Liquid-based scented material 32 is applied to the pad 24. Typically, such scented material 32 is provided in a bottle 34 so that the user can apply the desired amount of scented material 32 to the pad 24 to increase or decrease the amount of scent within the air. Also, the pad 24 may occasionally need to be refreshed with scented material 32. Of course, bottles 34 of different scented material 32, such as vanilla, potpourri, cologne, etc. can be provided within the kits.

Typically, a tab 36 extends from a rear portion of the housing 12. The tab 36 includes an aperture 38 to facilitate attachment to the electrical device 28. Often, electrical devices 28, such as computers, include apertures around the fan outlet 26 such that a screw can be inserted through the aperture 38 of the tab 36 and into the electrical device 28 for securing the device 10 to the fan outlet 26. Alternatively, a hook 40 is provided which can be adhered, such as by double-sided tape or adhesive, to the electrical device 28. The tab 36 is then attached to the hook 40 by inserting the hook 43 of the aperture 38 allowing the device 10 to be suspended over the fan outlet 26, as shown in FIG. 8.

It will be appreciated that the odorizer/deodorizer device 10 of the present invention provides enhanced disbursement of scent throughout the area in which the electrical device 28 is located. The device 10 of the present invention operates in a convenient and concealed location without occupying an electrical outlet nor acting as a visible distraction within the room. The desired air freshening scent or deodorizer and be periodically changed by changing the scented insert 24, or applying different liquid-based scented material 32 to the absorbent pad insert 24. The housing 12 and other components of the invention can be reused throughout the life of the device 10.

Although several embodiments have been described in detail for purposes of illustration, various modifications may be made without departing from the scope and spirit of the invention. Accordingly, the invention is not to be limited, except as by the appended claims.

What is claimed is:

1. An odorizer/deodorizer device, comprising:
a housing defining an internal cavity, the housing having vents to allow air to be forced therethrough, and an end of the housing being configured such so as to be securely positioned in alignment with a fan outlet of an electrical device; and
a scented insert disposed within the housing, whereby air is forced through the housing by the fan to odorize or deodorize the air.

2. The device of claim 1, wherein the housing comprises two shells removably attachable to one another.

3. The device of claim 2, wherein the two shells are threadably attached to one another.

4. The device of claim 1, wherein opposing ends of the housing in alignment with the fan outlet are comprised of a mesh material.

5. The device of claim 1, including a tab extending from the housing and including an aperture for connection to the fan or electrical device.

6. The device of claim 5, including a hook attachable to the fan or electrical device and insertable through the aperture of the tab for positioning the housing relative to the fan.

7. The device of claim 1, wherein the insert comprises an absorbent pad containing a liquid-based scent material.

8. The device of claim 1, wherein the insert includes a plurality of apertures to facilitate the flow of air therethrough.

9. A method of odorizing/deodorizing an area, comprising the steps of:
placing a scented insert into a housing having vents; and
securely positioning the housing in alignment with a fan outlet of an electrical device, whereby air is forced through the housing by the fan and odorized or deodorized.

10. The method of claim 9, wherein the insert comprises an absorbent pad.

11. The method of claim 10, including the step of adding a liquid-based scent material to the pad.

12. An odorizer/deodizer kit, comprising:
a housing defining an internal cavity, the housing having vents to allow air to be forced therethrough, and an end of the housing being configured such so as to be securely positioned in alignment with a fan of an electrical device;
an absorbent pad configured to be disposed within the internal cavity of the housing; and
a bottle of liquid scent material.

13. The device of claim 12, wherein the housing comprises two shells removably attachable to one another.

14. The device of claim 13, wherein the two shells are threadably attached to one another.

15. The device of claim 12, wherein opposing ends of the housing in alignment with the fan are comprised of a mesh material.

16. The device of claim 12, including a tab extending from the housing and including an aperture for connection to the fan or electrical device.

17. The device of claim 16, including a hook attachable to the fan or electrical device and insertable through the aperture of the tab for positioning the housing relative to the fan.

18. The device of claim 12, wherein the pad includes a plurality of apertures to facilitate the flow of air therethrough.
